(19) United States
(12) Patent Application Publication
(10) Pub. No.: US 2023/0260109 A1
(43) Pub. Date: Aug. 17, 2023

(54) TUMOUR IMAGING DEVICE

(71) Applicant: INSTITUT NATIONAL DE RECHERCHE EN INFORMATIQUE ET EN AUTOMATIQUE, Le Chesnay Cedex (FR)

(72) Inventors: Irène VIGNON-CLEMENTEL, Le Chesnay Cedex (FR); Dirk DRASDO, Le Chesnay Cedex (FR); Yi YIN, Le Chesnay Cedex (FR)

(21) Appl. No.: 18/003,171

(22) PCT Filed: Jun. 24, 2021
(86) PCT No.: PCT/FR2021/051168

§ 371 Date: Dec. 23, 2022

(30) Foreign Application Priority Data
Jun. 26, 2020 (FR) FR2006763

(51) Int. Cl. G06T 7/00; G06T 3/40; G06T 7/11; G06T 7/60; A61B 5/055; G01R 33/563; G01R 33/56

(52) U.S. Cl. CPC G06T 7/0012; G06T 3/4007; G06T 7/11; G06T 7/60; A61B 5/055; G01R 33/56341; G01R 33/5608; G06T 2207/20021; G06T 2207/30096; G06T 2207/10088

(57) ABSTRACT

An imaging device includes: a memory, an upsampler, a slicer a selector and a guide. The memory is arranged to receive imaging data, biopsy data including needle data defining dimensions of a puncture carried out by means of a biopsy needle, and procedure data. The upsampler is arranged to upsample a raster image into a processing image. The slicer is arranged to partition the processing image from the upsampler into a set of regions. The selector is arranged to determine within the set of diffusion parameters a subset of diffusion parameters, and to return a subset of regions derived from the set of regions. The guide is arranged to determine, from the subset of regions, puncture parameter set data a puncture orientation and a puncture entry point and defining with the needle data a puncture zone.

### CROSS-REFERENCE TO RELATED APPLICATIONS

This Application is a Section 371 National Stage Application of International Application No. PCT/FR2021/051168, filed Jun. 24, 2021, the content of which is incorporated herein by reference in its entirety, and published as WO 2021/260334 on Dec. 30, 2021, not in English.

### FIELD OF THE DISCLOSURE

The present invention relates to the field of medical imaging, and more particularly to tumour imaging. Tumour imaging encompasses methods for non-invasive analysis of tumours. Analyses may be aimed at determining the extent of cancer cells within the tumour. Tumour imaging is carried out by magnetic resonance imaging (MRI), and in particular by diffusion-weighted magnetic resonance imaging. However, these two imaging techniques do not currently provide precise information about a tumour in a patient's body, such as the absolute distribution of cancer cells or the tumour load. By tumour load, it is meant here the total number of tumour cells of all types or each type individually, in particular the cancer cell type.

### BACKGROUND OF THE DISCLOSURE

Biopsies are known to be used as an invasive method to supplement or replace tumour imaging. A biopsy consists in locally sampling tissue by a biopsy needle. Biopsy provides local information about the tumour that is not accessible by non-invasive techniques. However, biopsy is a local sampling, and the tissue sampled is usually not representative of the tumour being studied. Furthermore, biopsy is an invasive surgical procedure and poses medical risks such as tumour enlargement, spread of cancer cells into healthy tissue, bleeding, infection or nerve damage.

There is no method to determine a representative location to perform a biopsy. As a result, it is currently necessary to carry out many biopsies, sometimes as many as ten, to increase the chances of obtaining enough information to determine information about the absolute distribution sought or the tumour load.

### SUMMARY

The invention improves the situation. To this end, the invention provides an imaging device comprising:
a memory, arranged to receive
   imaging data comprising at least one raster image derived from a cross-sectional diffusion-weighted magnetic resonance image of a tumour, each pixel of said raster image being associated with a diffusion parameter whose value represents the mobility of water molecules within said tumour,
   biopsy data comprising needle data defining the dimensions of a puncture carried out by means of a biopsy needle, and
   procedure data comprising a number of punctures to be performed in a biopsy procedure greater than or equal to 2 and a set of constraints associated with interventional constraints to be met to achieve the number of punctures to be performed,
an upsampler, arranged to upsample the raster image into a processing image whose spatial resolution is such that each pixel of the processing image corresponds to a region of the substantially square cross-sectional image whose side has a length less than a needle diameter dimension of the needle data,
a slicer arranged to partition the processing image from the upsampler into a set of regions wherein each region
   comprises pixels of the processing image directly adjoining each other at least in twos, is associated with a diffusion parameter whose value is substantially equal to the mean of the values of the diffusion parameters associated with its constituent pixels,
   the variance of the values of the diffusion parameters associated with the pixels of the region is less than a given variance value,
   corresponds to a portion of the cross-sectional diffusion-weighted magnetic resonance image of said tumour whose dimensions are greater than or substantially equal to the dimensions of a puncture carried out by means of a biopsy needle of the biopsy data,
      and wherein the diffusion parameters associated with each region of the set of regions form a set of diffusion parameters,
a selector, arranged to
   determine within the set of diffusion parameters a subset of diffusion parameters comprising as many diffusion parameters as the number of punctures to be performed of the procedure data, the subset of diffusion parameters comprising at least a first diffusion parameter selected from the first decile of the diffusion parameter values of the set of diffusion parameters, a second diffusion parameter from the last decile of the diffusion parameter values of the set of diffusion parameters, and, when the number of punctures to be performed of the procedure data is greater than 2, diffusion parameters whose respective diffusion parameter values are between the value of the first diffusion parameter and the value of the second diffusion parameter,
return a subset of regions derived from the set of regions and wherein each region is associated with a diffusion parameter substantially equal to one of the parameters of the subset of diffusion parameters,
a guide, arranged to determine, from the subset of regions, puncture parameter set data equal in number to the number of punctures to be performed of the procedure data, each set of puncture parameters comprising a puncture depth, a puncture orientation and a puncture entry point and defining with the needle data a puncture zone which is substantially included in one of the regions of the subset of regions, such that the sets of puncture parameters together meet the sets of constraints of the procedure data.

This imaging device is very advantageous, as it makes it possible to automatically determine a reduced number of biopsy locations ensuring relevant information. Indeed, the punctures determined by the imaging device are much more representative of the entire tumour than randomly taken punctures, while at the same time being fewer in number.

In various alternatives, the device may have one or more of the following characteristics:
the upsampler is arranged to upsample the raster image into the processing image by carrying out an interpolation,
the upsampler is arranged to carry out a bicubic interpolation to upsample the raster image into the processing image,
the slicer is arranged to partition the processing image from the upsampler by a super-pixel method,
the number of punctures to be performed in a biopsy procedure of the procedure data stored in the memory is less than or equal to 4,
the selector selects the first diffusion parameter from the second percentile of the diffusion parameter values of the set of diffusion parameters, and the second diffusion parameter from the ninety-eighth percentile of the diffusion parameter values of the set of diffusion parameters,
the first diffusion parameter and the second diffusion parameter are each equal to the median value of their respective quantile,
when the number of punctures to be performed of the procedure data is greater than 2, the selector chooses the diffusion parameters of the subset of diffusion parameters so that they are equidistant from each other in twos,
when the number of punctures to be performed of the procedure data is greater than 2, the selector determines a median value of the diffusion parameters of the set of diffusion parameters, and determines the diffusion parameters, j being a natural number comprised between 1 and the number of punctures to be performed of the procedure data, of the subset of diffusion parameters according to the equation:

Dj=Dini+Delta(j−1),

where Dini satisfies the equation:

Dini=M−min(|M−Dmin|,|M−Dmax|)

and Delta satisfies the equation:

Delta=2(M−Dini)/(N−1),

Dmin being equal to the value of the first diffusion parameter determined by the selector and Dmax being equal to the value of the second diffusion parameter of the subset of diffusion parameters determined by the selector,
the puncture depth, the puncture orientation, the puncture entry point of each of the sets of puncture parameters determined by the guide define with the biopsy data a puncture trajectory of the biopsy needle, and wherein the set of constraints of the procedure data stored in the memory comprises one or more constraints selected from the group comprising:
the punctures of a biopsy procedure have to have the same entry point into the tumour,
the puncture trajectories are in a straight line and have to be within a maximum puncture angle,
the puncture depth is less than a maximum puncture depth,
the puncture zone is spaced from the edges of the tumour by a predetermined distance,
if the tumour comprises a necrotic zone, the puncture trajectory does not penetrate said necrotic zone,
if the tumour comprises a fatty tissue zone, none of the puncture zones intersects said fatty tissue zone,
at least one of the entry points of the puncture parameter set data is fixed in advance,
the memory is further arranged to receive puncture data comprising a set of cell densities, each cell density being associated with one of the puncture zones of the puncture parameter set data, each cell density having a value representing a cell density of said puncture zone,
the imaging device further comprising:
a correlator, arranged to associate with each cell density value associated with one of the puncture zones the diffusion parameter value of the region in which said puncture zone is substantially included, for the set of regions, and to derive correlation data associating cell density values and diffusion parameter values of the set of diffusion parameters, and
a constructor, arranged to determine density data from the processing image and the correlation data, the density data comprising a density image of which each pixel, which corresponds to a region of the cross-sectional image, is associated with a cell density whose value is associated in the correlation data with the diffusion parameter value of a pixel of the processing image corresponding to said region of the cross-sectional image of said pixel.
the correlator carries out a strictly monotonic linear or non-linear interpolation on the basis of pairs formed of a diffusion parameter value and a cell density value associated with a puncture zone and derives the correlation data from the result of said interpolation,
the cell density of the puncture data and the density data corresponds to a total cell density or a cancer cell density,
the device further comprises an estimator arranged to determine a tumour load of the tumour based on the density data.

### BRIEF DESCRIPTION OF THE DRAWINGS

Further characteristics and advantages of the invention will be set out in detail in the following description, made with reference to the attached drawings, in which:

FIG. 1 represents a schematic view of the imaging device according to the invention,

FIG. 2 represents a raster image of a tumour obtained by diffusion-weighted magnetic resonance received by the imaging device of FIG. 1,

FIG. 3 represents a schematic view of a set of constraints received by the imaging device of FIG. 1,

FIG. 4 represents a processing image obtained by the imaging device of FIG. 1,

FIG. 5 represents a set of regions determined by the imaging device of FIG. 1,

FIG. 6 represents a set of diffusion parameters determined by the imaging device of FIG. 1,

FIG. 7 represents sets of puncture parameters determined by the imaging device of FIG. 1,

FIG. 8 represents a subset of regions determined by the imaging device of FIG. 1,

FIG. 9 represents regions of the subset of regions in FIG. 7,

FIG. 10 represents another subset of regions determined by the imaging device of FIG. 1,

FIG. 11 represents a detail of FIG. 10,

FIG. 12 represents an alternative to the imaging device of FIG. 1,

FIG. 13 represents a first linear interpolation carried out by the imaging device of FIG. 12,

FIG. 14 represents a second linear interpolation carried out by the imaging device of FIG. 12,

FIG. 15 represents a third linear interpolation carried out by the imaging device of FIG. 12,

FIG. 16 represents a tumour load estimate carried out by the imaging device of FIG. 12,

FIG. 17 represents tumour loads from FIG. 16 determined by the imaging device of FIG. 12,

FIG. 18 represents tumour loads measured for randomly taken punctures meeting the set of constraints.

### DETAILED DESCRIPTION OF ILLUSTRATIVE EMBODIMENTS

The appended drawings contain, for the most part, elements of certainty. They may therefore not only serve to better understand the present invention, but also contribute to its definition, where appropriate.

The description is followed by an Appendix A comprising mathematical formulae. This appendix forms an integral part of the description.

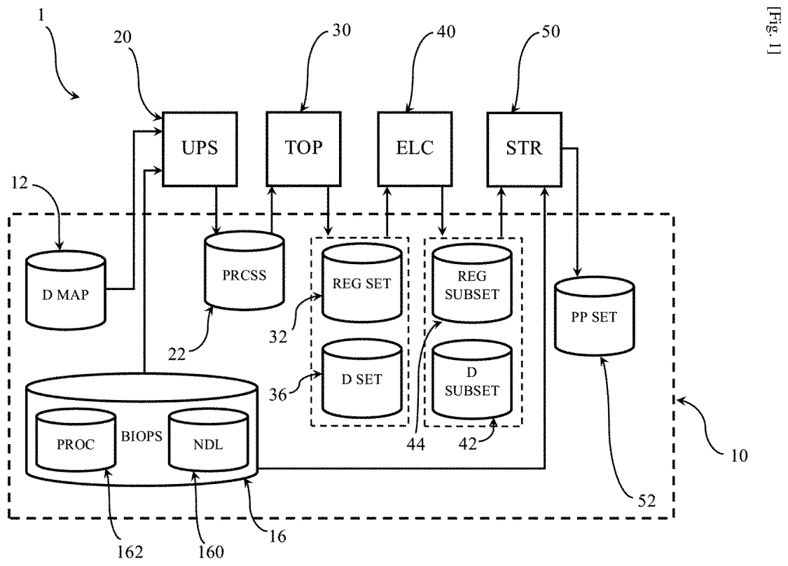

Reference is made to FIGS. 1 to 11.

An imaging device 1 according to the invention comprises a memory 10, an upsampler 20, a slicer 30, a selector 40 and a guide 50.

The memory 10 may be any type of data storage suitable for receiving digital data: hard disk, flash memory hard disk (SSD), flash memory in any form, random access memory, magnetic disk, locally or cloud distributed storage, etc. The data calculated by the device 1 can be stored on any type of memory similar to or on the memory 10. This data can be erased after the device has performed its tasks or retained.

The upsampler 20, the slicer 30, the selector 40 and the guide 50 are here programs run by the computer processor. Alternatively, one or more of these elements could be implemented in a different way by means of a dedicated processor. By processor, it has to be understood any processor adapted to data processing as described below. Such a processor may be realised in any known way, in the form of a microprocessor for a personal computer, a dedicated chip of the FPGA or SoC type, a computing resource on a grid, a microcontroller, or any other form suitable to provide the computing power necessary for the embodiment described below. One or more of these elements may also be realised in the form of specialised electronic circuits such as an ASIC. A combination of processors and electronic circuits may also be contemplated.

The memory 10 receives imaging data 12, comprising at least one raster image 14 from a cross-sectional diffusion-weighted magnetic resonance imaging (diffusion MRI or DWI) image of a tumour 200. The raster image 14 is also known as a “D-map”. Diffusion-weighted magnetic resonance is a tissue imaging technique consisting in measuring the diffusion of water molecules within the tissue. Each pixel of the raster image 14 is thus associated with a diffusion parameter whose value represents the mobility of water molecules within the tissue of the tumour 200. The higher the value of the diffusion parameter, the greater the diffusion of water molecules, or, in other words, the freer the water molecules are to move within the tissue.

Diffusion parameter values are highly dependent on the diffusion-weighted magnetic resonance imaging machine that generates them, which generally makes it impossible to analyse them without additional information. In the example described here, the raster image 14 represented in FIG. 2 has pixels with a side corresponding to a substantially square zone of 2.1 mm on a side.

The applicant's work has shown that the diffusion value is correlated with the cell density. Thus, starting from a raster image 14 derived from a cross-sectional diffusion-weighted magnetic resonance image, it would in theory be possible to observe cellular heterogeneities within the tumour, at least qualitatively. However, the resolution of the raster images provided by current diffusion-weighted magnetic resonance imaging devices is too low to represent such heterogeneities in a usable way, so that they cannot be used in practice, except to carry out a full tumourectomy to see the correspondence. A systematic tumourectomy is of course out of the question (and in most cases impossible), especially since if this were possible, imaging and biopsies would be useless.

In one embodiment, the imaging data 12 comprises a plurality of raster images 14 representing successive cross-sectional images of the tumour 200. This provides a three-dimensional view of the tumour 200. In the embodiment described here, the imaging data 12 comprises 9 raster images 14. In another embodiment, the imaging data 12 comprises 40 raster images 14 from which 9 raster images 14 representing sections closest to a median plane of the tumour 200 are derived. One of these raster images 14 is represented in FIG. 2, the scale on the right-hand side of the figure indicating a correspondence between pixel intensity and diffusion parameter value.

The memory 10 also receives biopsy data 16, comprising needle data 160 and procedure data 162.

The needle data 160 defines the dimensions of a puncture carried out with a biopsy needle. In the embodiment described here, the dimensions of the puncture comprise a puncture diameter and a puncture length, and the sampling with the biopsy needle is a substantially cylindrical puncture zone defined by that diameter and length. In the example described here, the puncture diameter is substantially 0.84 mm, and the puncture length is 4.55±1.45 mm.

The procedure data 162 comprises a number of punctures to be performed in a biopsy procedure. The number of punctures to be performed is an integer greater than or equal to 2. Indeed, it is necessary to perform at least two punctures to obtain sufficient information about the tumour 200. In the embodiment described here, the number of punctures is comprised between 2 and 4, inclusive.

The procedure data 162 also comprises a set of constraints 300. The set of constraints defines interventional constraints to be met in a biopsy procedure to carry out one or more punctures. The constraints in the sets of constraints may arise from the type of organ in which the tumour 200 is located, the size of the tumour 200, the presence of necrotic tissue 500 within the tumour 200, or any other parameter constraining the performance of the biopsies.

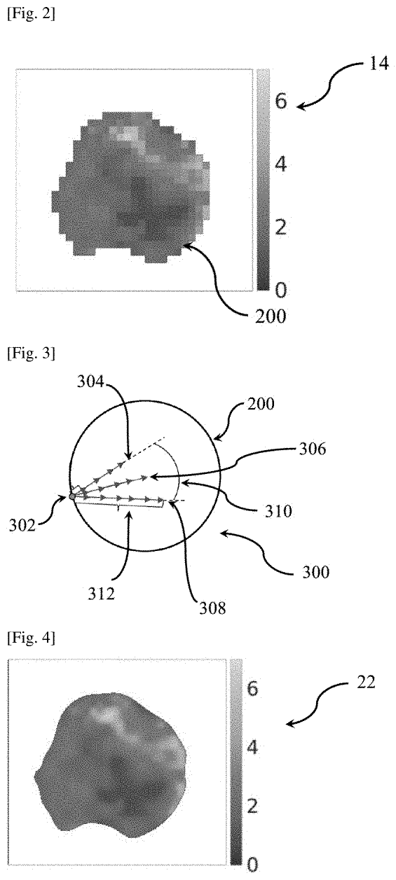

In the example described here, the tumour 200 is a lung tumour, and the biopsy constraints associated thereto, represented in FIG. 3, are as follows:
the different biopsies all have to have the same entry point 302 into the tumour 200,
the different needle trajectories 304, 306, 308 are in a straight line and have to be within a maximum puncture angle 310,
the depth at which a biopsy needle is inserted into the tumour is less than a maximum puncture depth 312.

In the example described here, the maximum puncture angle 310 is 20° and the maximum puncture depth 312 is 2.2 cm.

Constraints may also include
the proximity of the zone punctured by a biopsy to the edges of the tumour, for example with punctures at least 2.5 mm from said edges,
the existence of necrotic tissue within the tumour, through which the needle should not pass during a biopsy,
the exclusion of fatty tissue from the zones in which puncture can be performed,
fixing a specific entry point or set of entry points at the edge of the tumour,
avoidance of the most vascularised zones to reduce the risk of diffusion of cancer cells,
the presence of bones that prevent puncture at certain entry points and angles, for example the ribs in the case of a lung tumour, and
the specifics of the organ and/or biopsy procedure being contemplated. For example, for transrectal ultrasound biopsy of the prostate, the patient is in the left lateral position, thus limiting accessibility.

As discussed above, the pixels each correspond to a region of the cross-sectional image that is substantially square in shape and 2.1 mm on a side, and the puncture zone for the puncture needle has a diameter of substantially 0.84 mm and a length of 4.55±1.45 mm. Thus, there are two obstacles to an analysis of the raster image 14:
1) On the one hand, its pixels are too large to represent tumour heterogeneities.
2) On the other hand, its pixels are too small for the biopsy needle to sample tissue in a puncture zone with certain diffusion parameter and homogeneity properties, substantially included in a region corresponding to a single pixel.

The upsampler 20 refines the raster image to overcome the first obstacle.

To do so, the upsampler 20 receives the raster image 14 and upsamples it into a processing image 22. The processing image 22 is an image of much higher spatial resolution than the raster image 14, as can be seen by comparing FIG. 2 and FIG. 4. Each of the pixels in the processing image 22 corresponds to a substantially square cross-sectional region of the image. The resolution of the processing image 22 is chosen by the upsampler 20 such that each of its pixels has a side length less than the puncture diameter of the needle data 160.

In the embodiment described here, the upsampler 20 applies an interpolation technique to upsample the raster image 14. The interpolation techniques used may be bicubic interpolation, bilinear interpolation, Bell filter, Hermite filter, Mitchell filter, Lanczos filter, or any other conventional interpolation technique adapted to images. In the example described here, the upsampler 20 applies the bicubic interpolation technique, described for example in Keys, R. (1981). “Cubic convolution interpolation for digital image processing.” IEEE transactions on Signal Processing. In Acoustics, Speech, and Signal Processing (Vol. 29, p. 1153).

Alternatively, the upsampler 20 may apply a Discrete Cosine Transform (DCT) technique to upsample the raster image 14, such as those described in Dugad, R., & Ahuja, N. (2001). “A fast scheme for image size change in the compressed domain.” IEEE Transactions on Circuits and Systems for Video Technology, 11(4), 461-474. and in Park, H., Park, Y., & Oh, S. K. (2003). “L/M-fold image resizing in block-DCT domain using symmetric convolution.” IEEE Transactions on Image Processing, 12(9), 1016-1034. Alternatively, a machine learning-based technique, in particular deep learning, such as that described in Wang, Z., Chen, J., & Hoi, S. C. (2019). “Deep learning for image super-resolution: A survey.” arXiv preprint arXiv:1902.06068, could be used.

The processing image 22 has pixels whose side corresponds to a substantially square zone with a side at least 5 times smaller than the side of the zone of a pixel of the raster image 14, preferably 20 times smaller, as in the example described here.

In another embodiment, the imaging device 1 works in three dimensions, and the imaging data 12 comprises 9 successive cross-sectional raster images 14, separated in twos by a distance of 6 mm. The raster images 14 thus define voxels of dimension 2.1 mm×2.1 mm×6 mm. The processing images 22 define voxels at least 5 times smaller in each dimension, preferably 20 times smaller. In the example described here, the voxels of the processing images 22 each define a zone of 0.105×0.105×0.3 mm.

The processing image 22 has a much higher resolution than the raster image 14. The regions corresponding to the pixels in the processing image are much smaller than the heterogeneities in the tumour. The pixels of the processing image 22 now have to be grouped together to slice the cross-sectional image into a plurality of regions sufficiently homogeneous to overcome the first obstacle and sufficiently large to overcome the second obstacle.

To do so, the slicer 30 receives and partitions the processing image 22 into a set of regions 32. Each of the regions 34 of the set of regions 32 comprises pixels of the processing image 22 that are directly adjoining, at least in twos.

The region 34 is associated with a diffusion parameter value substantially equal to the mean of the diffusion parameter values of its constituent pixels. This diffusion parameter value of the region 34 may be chosen, for example, as the mean value of the diffusion parameter values associated with the pixels of the region 34, or as the median value.

The slicer 30 determines the set of regions 32 such that, for each region 34, the variance of the diffusion parameter values associated with the pixels of that region 34 is less than a threshold value. The regions 34 of the set of regions 32 are thus sets of contiguous pixels of the processing image 22 whose associated diffusion parameter values are substantially homogeneous, or, in other words, whose variance is low.

The slicer 30 further determines the set of regions 32 such that the region 34 corresponds to a portion of the cross-sectional diffusion-weighted magnetic resonance image whose dimensions are greater than or substantially equal to the dimensions of a puncture carried out by means of a biopsy needle of the needle data 160. Here, the portions to which the regions 34 correspond have substantially similar dimensions, with for example substantially equal respective areas. The diffusion parameters associated with each region 34 of the set of regions 32 form a set of diffusion parameters 36.

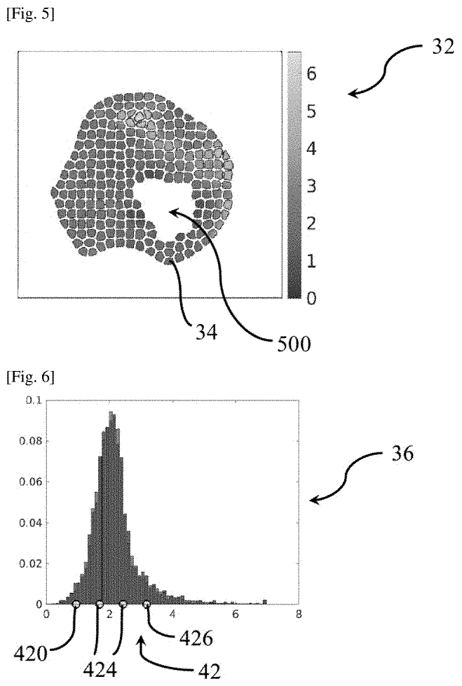

In one embodiment, the slicer 30 partitions the processing image 22 into the set of regions 32 by a super-pixel method with constraints on the size and low variance of the diffusion parameter values associated with the pixels of each region 34. In the example described here, the super-pixel method used is that described in Ren, X., & Malik, J. (2003, October). “Learning a classification model for segmentation.” In Proceedings Ninth IEEE International Conference on Computer Vision. (p. 10). IEEE. In the example described here, represented in FIG. 5, the super-pixels correspond to portions of the cross-sectional diffusion-weighted magnetic resonance image with a size slightly larger than the portions corresponding to the pixels of the raster image 14. Alternatively, one of the super-pixel methods described in Stutz, D., Hermans, A., & Leibe, B. (2018). “Super-pixels: An evaluation of the state-of-the-art.” Computer Vision and Image Understanding, 166, 1-27, could be used.

In FIG. 5, a part of the cross-sectional diffusion-weighted magnetic resonance image is not taken into account, as it is a region 500 corresponding to necrotic tissue. The determination of the data corresponding to the region 500 may be carried out by the upsampler 20, the slicer 30 or by another part of the imaging device 1. Alternatively, the data corresponding to the region 500 could be determined by an operator, for example before the biopsy operation is performed.

Alternatively, the slicer 30 may partition the processing image 22 into the set of regions 32 by a convex polygon partitioning method as described in Duan, L., & Lafarge, F. (2015). “Image partitioning into convex polygons.” In Proceedings of the IEEE Conference on Computer Vision and Pattern Recognition (pp. 3119-3127). This method is based on the construction of a Voronoi diagram whose cells are convex polygons by conforming to straight lines already drawn within the processing image 22. Here, regions 34 can be obtained whose dimensions can be controlled, and which preserve the edges of shapes or objects within the processing image 22 at a subpixel scale. The set of regions 32 here provides a representation of the heterogeneities within the processing image 22.

In the three-dimensional case, where several processing images 22 are produced by the upsampler 20, the slicer 30 partitions the processing images 22 using a super-voxel method, such as one described in the paper Xu, C., & Corso, J. J. (2012, June). “Evaluation of super-voxel methods for early video processing.” In 2012 IEEE conference on computer vision and pattern recognition (pp. 1202-1209). IEEE.

The regions 34 determined by the slicer 30 are thus
sufficiently large that a needle puncture zone can be substantially included in one of the regions 34, and
sufficiently homogeneous that a sampling within a region 34 is representative of that region 34.

The selector 40 determines, from the regions 34, which are relevant to a sample, so that the diffusion parameter values in those regions are representative of all diffusion parameters.

To do so, the selector 40 determines within the set of diffusion parameters 36 a subset of diffusion parameters 42 which comprises as many diffusion parameters as the number of punctures to be performed of the procedure data 162. Since the number of punctures to be performed is at least two, the subset of diffusion parameters 42 comprises at least two diffusion parameters. A first diffusion parameter 420 of the diffusion parameters is selected from the first decile of the diffusion parameter values of the set of diffusion parameters 36. A second diffusion parameter 426 of the diffusion parameters is selected from the last decile of the diffusion parameter values of the set of diffusion parameters 36. The first diffusion parameter 420 and the second diffusion parameter 426 may for example each be the median value of their respective decile. This ensures that the subset of diffusion parameters 42 determined by the selector 40 is representative of the diffusion parameter values of the regions 34 of the set of regions 32, in particular the extreme values of the diffusion parameters of the set of diffusion parameters 36.

In one embodiment, the first diffusion parameter 420 and the second diffusion parameter 426 are selected from the 5-th and 95-th percentile of the set of diffusion parameters, respectively, preferably from the 2-th and 98-th percentile. Here, the first diffusion parameter 420 and the second diffusion parameter 426 are each the median value of their respective quantile.

When the number of punctures to be performed of the procedure data 162 is greater than 2, the selector 40 determines additional diffusion parameters 424 in addition to the first diffusion parameter 420 and the second diffusion parameter 426. The respective values of these additional diffusion parameters 424 are between the value of the first diffusion parameter 420 and the value of the second diffusion parameter 426.

In one embodiment, the selector 40 chooses the diffusion parameters 424 comprised between the first diffusion parameter 420 and the second diffusion parameter 426 such that the diffusion parameters of the subset of diffusion parameters 42 have values that are equidistant from each other in twos. To do so, the selector 40 applies the equation (1) in Appendix A, where Dmin is the value of the first diffusion parameter 420, Dmax is the value of the second diffusion parameter 426, N is the number of punctures to be performed in a biopsy procedure of the procedure data 162, j is an integer between 1 and the number of punctures to be performed inclusive, and Dj is the value of the j-th diffusion parameter of the subset of diffusion parameters 42. The subset of diffusion parameters 42 thus obtained is represented in FIG. 6, where the axis x indicates the diffusion parameter values and the axis y indicates the probability of having a diffusion parameter value. The subset of diffusion parameters 42 comprises few diffusion parameters (4, in the example described here), but these are highly representative of all diffusion parameters in the set of diffusion parameters 36, including for extreme values.

Alternatively, when the set of diffusion parameters 36 has a distribution of its diffusion parameters around the median value M of the diffusion parameters of the set of diffusion parameters 36 that is highly skewed, the selector 40 determines the diffusion parameters 424 according to formula (2) of Appendix A, where Dini is determined according to formula (3) of Appendix A and Delta is determined according to formula (4) of Appendix A. This alternative allows less weight to be given to the longest tail of the diffusion parameter distribution of the set of diffusion parameters 36, such that the subset of diffusion parameters 42 is representative of the diffusion parameter distribution of the set of diffusion parameters 36. This skewness may, for example, occur if there is too much noise in the raster image 12, which disturbs small diffusion parameter values more than high diffusion parameter values. The determination of this skewness, and thus the choice of this skewed method or the equidistance method described above, can be carried out manually, or automatically by the selector 40.

In one embodiment, the selector 40 calculates the skewness coefficient of the set of diffusion parameters 36. If this skewness coefficient exceeds a certain threshold, the selector 40 determines the diffusion parameters 424 according to formula (2). Otherwise, the selector 40 applies formula (1).

The selector 40 returns a subset of regions 44 comprising regions 46 derived from the set of regions 32. Each region 46 of the subset of regions 44 is associated with a diffusion parameter substantially equal to one of the diffusion parameters of the subset of diffusion parameters 42. Furthermore, in the example described here, all of the diffusion parameters are each associated with at least one of the regions 46. Alternatively, for each diffusion parameter of the subset of diffusion parameters 42, the subset of regions 44 may comprise all of the regions 34 of the set of regions 32 associated with said diffusion parameter of the subset of diffusion parameters 42. In the embodiment where the set of diffusion parameters is sliced into quantiles, the diffusion parameter of each region 46 of the subset of regions 44 is included in the same quantile as the diffusion parameter of the associated subset of diffusion parameters 42.

The subset of regions 44 comprises regions 46 having diffusion parameter values well distributed in the set of diffusion parameters 36. In fact, a sampling from a plurality of these regions 46 with diffusion parameter values being different in twos is representative of all regions 34.

However, the tumour 200 has a number of features, such as the shape of the set of constraints 300 or the presence of necrotic regions 500. Based on these features, the guide 50 determines which regions of the subset of regions 44 should be sampled, and how to perform the sampling.

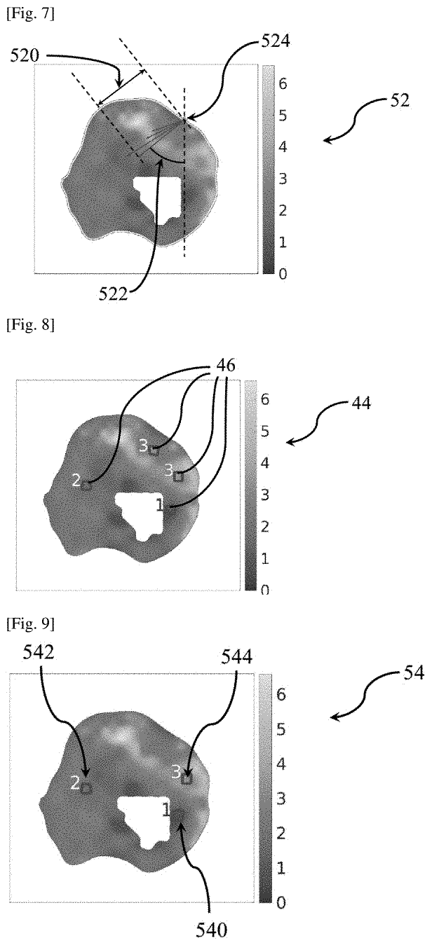

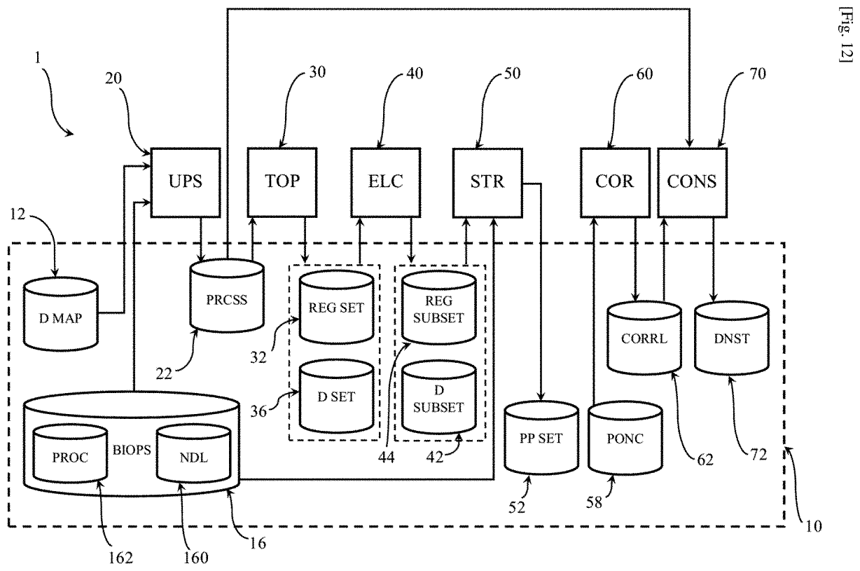

To do so, the guide 50 determines from the subset of regions 44, puncture parameter set data 52, equal in number to the number of punctures to be performed of the procedure data 162. Each set of puncture parameters of the puncture parameter set data 52 comprises a puncture depth 520, a puncture orientation 522 and a puncture entry point 524, represented in the example described here in FIG. 7. Each set of puncture parameters defines with the needle data 160 a puncture zone 526, visible in FIG. 11, which is substantially included in one of the regions 46 of the subset of regions 44. The guide 50 determines the sets of puncture parameters of the puncture parameter set data 52 such that the sets of puncture parameters together meet the sets of constraints 300 of the procedure data 162.

In the example represented in FIG. 8, the guide 50 receives a subset of regions 44 from the selector 40 comprising four regions 46. Here, the subset of diffusion parameters 42 comprises three diffusion parameters P1, P2 and P3. The diffusion parameters P1 and P2 are each associated with a single region of the subset of regions 44 respectively referenced as “1” and “2” in FIG. 8. The parameter P3 is associated with two regions of the subset of regions 44 referenced “3” in FIG. 8. The guide 50 then selects, from these regions 46, a set of regions 54, represented in FIG. 9, adapted to a puncture as a function of the sets of constraints 300. As the procedure involves three biopsies, this set of regions 54 includes three regions 540, 542 and 544. Regions 540 and 542 correspond to parameters P1 and P2, respectively. Region 544 labelled “3” is selected from one of the two regions corresponding to the diffusion parameter P3 in FIG. 8.

In some cases, the guide 50 may determine that the subset of regions 44 is incompatible with the set of constraints 300. This means that it is not possible to perform as many punctures as originally intended meeting the sets of constraints 300. In this case, the selector 40 is run to determine a new subset of diffusion parameters 42 comprising one less diffusion parameter than previously, and then a subset of regions 44. This is repeated until the guide 50 determines a subset of regions 44 compatible with the set of constraints 300, or it is determined that the biopsy procedure is not possible.

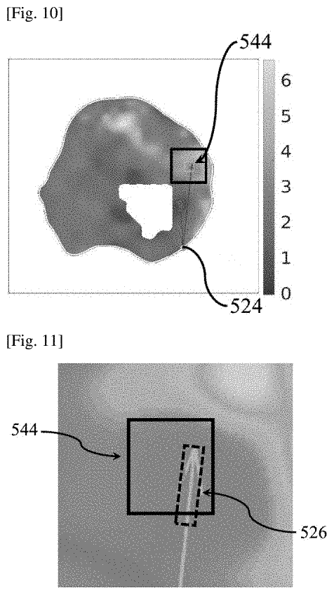

In another example, represented in FIG. 10 and FIG. 11, the new set of regions 54 selected by the guide 50 includes three regions 540, 542 and 544. Here, the puncture zone 526 of the puncture parameter set data 52 determined by the guide 50 is substantially included in the region 544 of the new set of regions 54.

The puncture parameter set data 52 determined by the guide 50 is provided to a surgeon or radiologist who is then able to carry out much more relevant biopsies than if he or she were to select them by hand or randomly. Furthermore, as the number of biopsies performed is small, less than 4 in the example described here, the medical risks are greatly reduced.

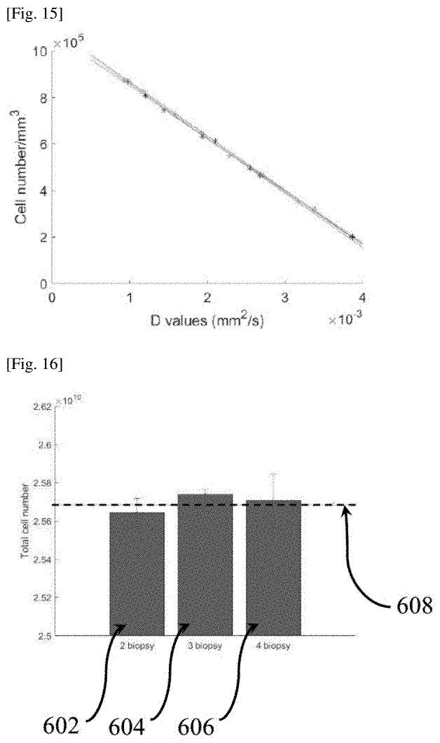

Reference is now made to FIGS. 12 to 15.

In the embodiment described here, the imaging device 1 retrieves the result of a biopsy procedure carried out according to the puncture parameter set data 52. This result is stored in the memory as puncture data 58. From the puncture data 58, the imaging device 1 reconstructs a density map of the tumour 200.

To do so, the imaging device 1 comprises a correlator 60 and a constructor 70. The correlator 60 derives from the puncture data 58 a correlation between the diffusion parameter values of the set of diffusion parameters 36 and cell density values in the tumour 200. The cell density may be a total cell density or a cancer cell density. The constructor 70 uses this correlation to reconstruct a cell density map within the tumour 200.

The puncture data 58 comprises a set of cell densities. Each cell density is associated with one of the puncture zones 526 of the puncture parameter set data 52. Each density has a value representing a cell density of the puncture zone 526 associated thereto.

This puncture data 58 may be obtained after a biopsy procedure by a surgeon or radiologist and after analysis of samples taken from the puncture zones 526 of the puncture parameter set data 52. The analysis of this puncture data 58 can be carried out by a histology method as described in Yin, Y., Sedlaczek, O., Müller, B., Warth, A., González-Vallinas, M., Lahrmann, B., Grabe, N., Kauczor, HU., Breuhahn, K., Vignon-Clementel, IE., Drasdo, D. (2018). “Tumour cell load and heterogeneity estimation from diffusion-weighted MRI calibrated with histological data: an example from lung cancer.” IEEE transactions on medical imaging, 37(1), 35-46.

The correlator 60 receives the puncture data 58 and associates with each cell density value associated with one of the puncture zones 526 the diffusion parameter value of the region 46 in which said puncture zone 526 is substantially included. The correlator 60 determines correlation data 62 that associates cell density values with diffusion parameter values of the set of diffusion parameters 36.

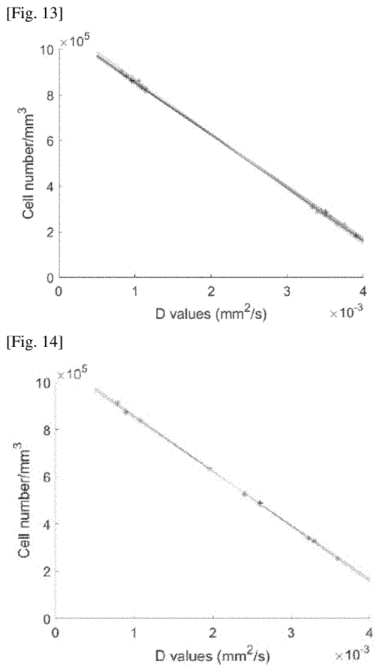

In the embodiment described here, the correlator 60 determines pairs each associating a diffusion parameter value and a cell density value associated with one of the puncture zones of the puncture parameter set data 52. Based on these pairs, the correlator 60 performs a linear interpolation, the result of which relates the diffusion parameter values of the set of diffusion parameters 36 to cell density values. The correlator 60 determines correlation data 62 based on the result of the linear interpolation. FIGS. 13, 14 and 15 represent linear interpolations for a number of punctures of the procedure data 162 of two, three and four respectively. FIGS. 13, 14 and 15 are fitted with an axis x indicating the diffusion parameter values, and an axis y indicating a cell density. The higher the diffusion parameter value, indicating high mobility of water molecules within the tissue, the lower the cell density.

Alternatively, the interpolation carried out by the correlator 60 may be a strictly monotonic polynomial interpolation, or other strictly monotonic function.

The constructor 70 determines density data 72 from the processing image 22 and the correlation data 62. The density data 72 comprises a density image. Each pixel of the density image corresponds to a region of the cross-sectional image to which a pixel of the processing image 22 also corresponds. The constructor 70 associates with each pixel of the density image the cell density value associated in the correlation data 62 with the diffusion parameter value of said pixel of the processing image 22.

Thus, from the raster image 14, the imaging device 1 determines here a reduced number of samplings to be carried out, and then, based on the result of these samplings, is able to reconstruct an accurate map of the cell density of the tumour 200.

Reference is made to FIG. 16. FIG. 16 is fitted with an axis x indicating a number of punctures performed, and an axis y indicating a tumour load value.

In one embodiment, not represented in the figures, the imaging device 1 further comprises an estimator arranged to determine a tumour load of the tumour 200. Here, the estimator carries out a numerical area integration over the entire area of the density image from which it derives the tumour load. FIG. 16 represents a tumour load 602, 604, 606 determined by the estimator, depending on whether the number of punctures is two, three or four, respectively, and compares this tumour load to a reference tumour load 608. The higher the number of punctures, the closer the tumour load determined by the estimator is to the reference tumour load 608. The imaging device 1 is thus able to measure a tumour load with only two biopsies, where conventional methods with randomly taken biopsies with 8 or even 10 biopsies are less accurate. With three or four biopsies, the measured tumour load is even more accurate, while keeping the number of punctures low.

Here, the imaging device 1 is thus able to determine the tumour load of the tumour 200 from the result of samplings according to the puncture parameter set data 52, and to visualise and/or quantify the tumour heterogeneity.

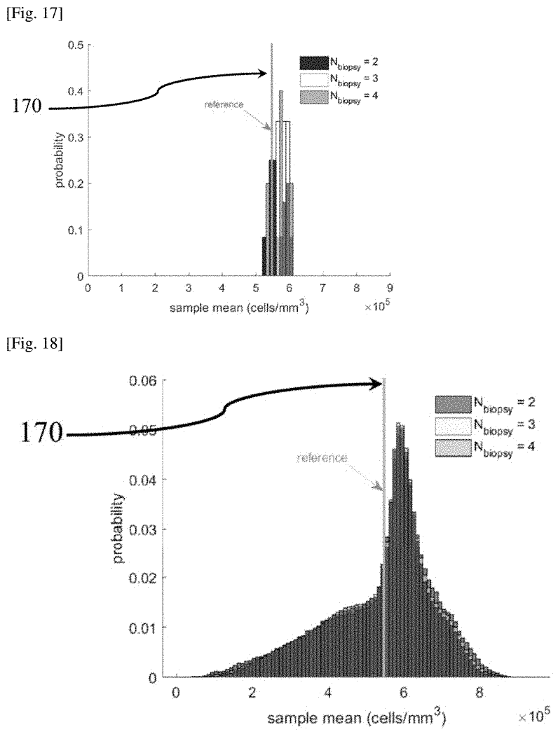

Reference is made to FIGS. 17 and 18. Each figure represents three histograms based on the determination of tumour loads from two, three and four biopsies. Specifically, the axis x indicates a tumour load value, and the axis y indicates the percentage of biopsies having a value of estimated tumour loads compared to all biopsies carried out. Thus, in FIG. 17, 10% of the biopsy procedures involving 2 biopsies result in an estimated tumour load of 600,000 cells per mm³. In these figures, a reference tumour load value 170 is also represented to indicate the ideal target value that should be estimated.

In order to show the interest of the invention compared to the existing methods, a histogram derived from biopsies selected by means of the device according to the invention is represented in FIG. 17, while FIG. 18 represents a histogram made from randomly carried out punctures meeting the set of constraints 300.

These figures show that the puncture parameter set data 52 determined by the imaging device 1 allows punctures to be carried out for determining a tumour load that is much closer to the actual tumour load than random punctures. These figures also show that the tumour load measurements made on the basis of the puncture parameter set data 52 have much less scatter than the measurements made with random punctures.

The use of puncture parameter set data 52 determined by the imaging device 1 therefore allows the correlator 60, the constructor 70 and the estimator to determine a much more reliable tumour load, that is, closer to the reference value 170.

The imaging device 1 allows a surgeon or radiologist to accurately and robustly measure a tumour load from a very small number of punctures, typically three to five times fewer than conventional procedures.

The correlator 60, the constructor 70 and the estimator are programs run by the computer processor. The alternatives described for the upsampler 20, the slicer 30, the selector 40 and the guide 50 can also be applied to the correlator 60, the constructor 70 and the estimator.

In the foregoing, the slicer 30 determines regions 34 having a size greater than or substantially equal to the puncture zone of a biopsy needle. Alternatively, the slicer 30 could determine regions 34 having dimensions less than the puncture length, but greater than the puncture diameter. For biopsies carried out on this basis, histological measurements would then be carried out only on the part of the samplings corresponding to the regions 540, 542, 544.

Alternatively, the device may also receive in memory 10 data from other types of non-invasive imaging whose pixels of the raster images 14 are associated, instead of the diffusion parameter in the case of DWI, with a parameter that reflects cell density.

### Appendix A

Dj=Dmin+(j−1)(Dmax−Dmin)/(N−1) (1)

Dj=Dini+Delta(j−1) (2)

Dini=M−min(|M−Dmin|,|M−Dmax|) (3)

Delta=2(M−Dini)/(N−1) (4)

Although the present disclosure has been described with reference to one or more examples, workers skilled in the art will recognize that changes may be made in form and detail without departing from the scope of the disclosure and/or the appended claims.

1. An imaging device comprising:
a memory, arranged to receive:
imaging data comprising at least one raster image derived from a cross-sectional diffusion-weighted magnetic resonance image of a tumour, each pixel of said raster image being associated with a diffusion parameter whose value represents the mobility of water molecules within said tumour,
biopsy data comprising needle data defining dimensions of a puncture carried out by means of a biopsy needle, and
procedure data comprising a number of punctures to be performed in a biopsy procedure greater than or equal to 2 and a set of constraints associated with interventional constraints to be met in order to carry out the number of punctures to be performed,
an upsampler, arranged to upsample the raster image into a processing image whose spatial resolution is such that each pixel of the processing image corresponds to a region of the substantially square cross-sectional image whose side has a length less than a needle diameter dimension of the needle data;
a slicer arranged to partition the processing image from the upsampler into a set of regions in which each region
comprises pixels of the processing image directly adjoining each other at least in twos, is associated with a diffusion parameter whose value is substantially equal to the mean of the values of the diffusion parameters associated with its constituent pixels,
the variance of the values of the diffusion parameters associated with the pixels of the region is less than a given variance value,
corresponds to a portion of the cross-sectional diffusion-weighted magnetic resonance image of said tumour whose dimensions are greater than or substantially equal to the dimensions of a puncture carried out by means of a biopsy needle of the biopsy data,
and wherein the diffusion parameters associated with each region of the set of regions form a set of diffusion parameters,
a selector, arranged to
determine within the set of diffusion parameters a subset of diffusion parameters comprising as many diffusion parameters as the number of punctures to be performed of the procedure data, the subset of diffusion parameters comprising at least a first diffusion parameter selected from the first decile of the diffusion parameter values of the set of diffusion parameters, a second diffusion parameter from the last decile of the diffusion parameter values of the set of diffusion parameters, and, when the number of punctures to be carried out of the procedure data is greater than 2, diffusion parameters whose respective diffusion parameter values are between the value of the first diffusion parameter and the value of the second diffusion parameter,
return a subset of regions derived from the set of regions and wherein each region is associated with a diffusion parameter substantially equal to one of the parameters of the subset of diffusion parameters,
a guide, arranged to determine, from the subset of regions, puncture parameter set data equal in number to the number of punctures to be performed of the procedure data, each set of puncture parameters comprising a puncture depth, a puncture orientation and a puncture entry point and defining with the needle data a puncture zone that is substantially included in one of the regions of the subset of regions, such that the sets of puncture parameters together meet the set of constraints of the procedure data.

2. The imaging device according to claim 1, wherein the upsampler is arranged to upsample the raster image into the processing image by carrying out an interpolation.

3. The imaging device of claim 2, wherein the upsampler is arranged to carry out a bicubic interpolation to upsample the raster image into the processing image.

4. The imaging device according to claim 1, wherein the slicer is arranged to partition the processing image from the upsampler by a super-pixel method.

5. The imaging device according to claim 1, wherein the number of punctures to be performed in a biopsy procedure of the procedure data stored in the memory is less than or equal to 4.

6. The imaging device according to claim 1, wherein the selector selects the first diffusion parameter from the second percentile of the diffusion parameter values of the set of diffusion parameters, and the second diffusion parameter from the ninety-eighth percentile of the diffusion parameter values of the set of diffusion parameters.

7. The imaging device according to claim 1, wherein the first diffusion parameter and the second diffusion parameter are each equal to the median value of their respective quantile.

8. The imaging device according to claim 1, wherein in response to the number of punctures to be performed of the procedure data being greater than 2, the selector chooses the diffusion parameters of the subset of diffusion parameters so that they are equidistant from each other in twos.

9. The imaging device according to claim 1, wherein in response to the number of punctures (N) to be performed of the procedure data being greater than 2, the selector determines a median value (M) of the diffusion parameters of the set of diffusion parameters, and determines the diffusion parameters (Dj), j being a natural number comprised between 1 and the number of punctures (N) to be performed of the procedure data, of the subset of diffusion parameters according to the equation:
Dj=Dini+Delta(j−1),
where Dini satisfies the equation:
Dini=M−min(|M−Dmin|,|M−Dmax|)
and Delta satisfies the equation:
Delta=2(M−Dini)/(N−1),
Dmin being equal to the value of the first diffusion parameter determined by the selector and Dmax being equal to the value of the second diffusion parameter of the subset of diffusion parameters determined by the selector.

10. The imaging device according to claim 9, wherein the puncture depth, the puncture orientation, the puncture entry point of each of the sets of puncture parameters determined by the guide define together with the biopsy data a puncture trajectory of the biopsy needle and wherein the set of constraints of the procedure data stored in the memory comprises one or more constraints selected from the group consisting of:
the punctures of a biopsy procedure have to have the same entry point into the tumour,
the puncture trajectories are in a straight line and have to be within a maximum puncture angle,
the puncture depth is less than a maximum puncture depth,
the puncture zone is spaced from the edges of the tumour by a predetermined distance,
if the tumour comprises a necrotic zone, the puncture trajectory does not penetrate said necrotic zone,
if the tumour comprises a fatty tissue zone, none of the puncture zones intersects said fatty tissue zone,
at least one of the entry points of the puncture parameter set data is fixed in advance.

11. The imaging device according to claim 1, wherein the memory is further arranged to receive puncture data comprising a set of cell densities, each cell density being associated with one of the puncture zones of the puncture parameter set data, each cell density having a value representing a cell density of said puncture zone,
the imaging device further comprising:
a correlator, arranged to associate with each cell density value associated with one of the puncture zones the diffusion parameter value of the region in which said puncture zone is substantially included, for the set of regions, and to derive correlation data associating cell density values and diffusion parameter values of the set of diffusion parameters, and
a constructor, arranged to determine density data from the processing image and the correlation data, the density data comprising a density image of which each pixel, which corresponds to a region of the cross-sectional image, is associated with a cell density whose value is associated in the correlation data with the diffusion parameter value of a pixel of the processing image corresponding to said region of the cross-sectional image of said pixel.

12. The imaging device according to claim 11, wherein the correlator carries out a strictly monotonic linear or non-linear interpolation based on pairs formed of a diffusion parameter value and a cell density value associated with a puncture zone and derives the correlation data from the result of said interpolation.

13. The imaging device according to claim 11, wherein the cell density of the puncture data and the density data corresponds to a total cell density or a cancer cell density.

14. The imaging device according to claim 11, further comprising an estimator arranged to determine a tumour load of the tumour based on the density data.

15. The imaging device of claim 1, wherein the upsampler, the slicer, the selector and the guide are implemented by at least one of:
a processor and a non-transitory computer readable medium storing a program, which when executed by the processor configures the processor to implement the up sampler, the slicer, the selector and the guide; or
an electronic circuit.
